(19) United States
(12) Patent Application Publication
(10) Pub. No.: US 2007/0038070 A1
(43) Pub. Date: Feb. 15, 2007

(54) Determining patient-related information on the position and orientation of MR images by the individualisation of a body model

(76) Inventor: Martin Tank, Heidelberg (DE)

Correspondence Address: SCHIFF HARDIN, LLP;PATENT DEPARTMENT, 6600 SEARS TOWER, CHICAGO, IL, 60606-6473, US

(21) Appl. No.: 10/575,032

(22) PCT Filed: Oct. 7, 2004
(86) PCT No.: PCT/DE04/02226

§ 371 Date: Apr. 7, 2006

(30) Foreign Application Priority Data
Oct. 7, 2003 (DE) 103 46 410.7

(51) Int. Cl. A61B 5/05

(52) U.S. Cl. 600/410

(57) ABSTRACT

In a method for determination of patient-related information regarding the position and orientation of slice image exposures in magnetic resonance tomographic examinations, initial MR overview exposures (magnetic resonance overview exposures) of the body of the patient are produced. Using these initial MR overview exposures, a predetermined, parameterized anatomical body model (i.e. an anatomical body model) with specific variable model parameters is then individualized (customized). The determination of the patient-related information about the position and orientation of the subsequent (diagnostic) slice image exposures then ensues on the basis of the relative position of the slice image exposures with regard to the individualized body model. In the individualization, by variation of the model parameters the body model is adapted to specific structures determined from the initial MR overview exposures, which specific structures advantageously represent the body surface of the patient. The individualization process thereby corresponds to a mathematical optimization problem. Those values of the variable model parameters are determined that minimize a deviation measure of the model relative to the structures from the overview exposures.

### BACKGROUND OF THE INVENTION

1 Field of the Invention

The present invention concerns a method for determination of patient-related (patient-referenced) information regarding the position and orientation of magnetic resonance tomographic slice image exposures of a patient.

2 Description of the Prior Art

In an examination of a patient in a magnetic resonance tomography apparatus (MR apparatus), among other things information about the position and orientation at which the respective exposures were made relative to the patient must be determined regarding the produced slice image exposures (exposures). Such patient-related information regarding the position and orientation of the slice image exposures is normally shown in a display or a printout of the exposures at the edges of the exposures, and enable a reconstruction of the spatial position of the exposures relative to the patient after the examination.

The acquisition plane in which a specific exposure is produced, in principle, can be defined within an arbitrarily-established reference coordinate system by the coordinates of directional vectors that span the appertaining acquisition plane. A uniform coordinate system, known as the “primary coordinate system” of the patient, is typically used as a reference coordinate system in radiological diagnostics. The axial directions of this primary coordinate system are defined by the intersection lines of the “primary planes” (standing perpendicular to one another) of the body of the patient. These primary planes are—as in anatomy—designated as transversal plane, sagittal plane and coronary plane. This is shown in FIG. 1, wherein the transversal plane is designated T, the sagittal plane is designated S and the coronary plane is designated C.

The precise orientation of a directional vector can be documented within this primary coordinate system of the patient, for example by two angle specifications with regard to the coordinate system axes. For the operator, however, such angle specifications generally can not be detected so quickly since the operator would then have to initially implement some conversions dependent on the reference coordinate system in order to acquire the position of the slice image exposure relative to the patient. Such angle specifications thus are not significant for daily practice.

For linguistic description of the patient-related orientation of arbitrarily-aligned exposures, letters or letter combinations are therefore generally used in radiology as what are known as “orientation markers”. Typical letters for use as orientation markers are:

A for “Anterior” (front)

P for “Posterior” (back)

L for “Left” (left)

R for “Right” (right)

H for “Head” (head-side)

F for “Feet” (foot-side)

These direction specifications are also indicated in FIG. 1 for clarification. Letter combinations such as, for example, “LP” for a combination made up of Left and Posterior can also be formed from these as further orientation markers. By these orientation markers, the position of the direction vectors spanning the acquisition plane is described relative to the primary coordinate system of the patient in the form of linguistic designations easily understandable for the health professional. The orientation markers are typically graphically represented on the edge of the exposure, as in a geographical map.

All such patient-related information regarding the position and orientation of the slice image exposures such as, for example, the specification of orientation markers in the primary coordinate system of the patient, must refer to a reference coordinate system which is oriented on the body of the patient. In contrast to this, in the data acquisition the coordinates of the individual volume elements from which the image information are acquired are determined in a fixed coordinate system with regard to the tomograph. The position of the patient must therefore remain known relative to the MR apparatus during the image data acquisition. Only under this condition can the desired patient-related information regarding the position and orientation of the slice image exposures in an magnetic resonance tomographic examination be definitely determined.

In the MR apparatuses typical today, the patient is inserted into the apparatus by a horizontally-movable patient bed and the position of the patient is determined by the operator of the MR apparatus. Differentiation is normally made between the head and foot positions as well as between the stomach, back, left-side and right-side positions. The operator typically has to select from among a number of possible patient positions from a selection list. A detailed description of the position of the patient, in particular a description of the arm position (for example whether the arm lies on the body or over the head) does not occur. After the specification of the patient position, the control software of the MR apparatus assumes that the patient is located in the described position in the normal position and consequently determines the primary coordinate system of the patient on the basis of this normal position. Given the production of an exposure, its orientation is then documented relative to this primary coordinate system. This means that, given the determination of the orientation markers that are ultimately graphically shown on the edge of the exposure for specification of the position of the exposure for the examining doctor, it is assumed that the patient is located in the normal position in the specific position.

An incorrect description of the patient position by the operator can thus lead by itself to incorrect orientation markers in the procedure typical today. The same problem occurs when the support of the patient does not correspond to the normal position. Moreover, it can also occur that correct orientation markers result again from an incorrect description of the patient position in combination with a support of the patient deviating from the normal position.

The accuracy demands for information regarding the position and orientation of the exposures can not be set too high. For example, an incorrect direction designation in a cranial exposure with a diagnosed brain tumor has fatal consequences for the operation planning. The operative entry could then be incorrectly selected. A correct determination of orientation markers is in particular very important in extremity exposures (arms and legs) and given symmetrical anatomy (for example in the cranium). The situation is particularly error-prone in examinations of the upper extremities. These can be positioned next to the body along the longitudinal body axis or be positioned over the head in the same direction, whereby an additional rotation movement of the hands relative to the elbow joint is still possible. This leads to a number of positioning possibilities that cannot practically be covered by all-inclusive position specifications. Conversely, for many examinations an individual patient positioning deviating from the positioning variants allowed by the system is absolutely reasonable or even essential in some cases. This exemplifies the need for an objective and standardized description of the patient position, which is an essential requirement for the correct determination of orientation markers.

In addition to the correct orientation determination, a reliable designation of the examination region is also important. For example, it must thus be ensured that it is unambiguously certain whether, for example, an examined extremity is the left or right extremity.

The determination of the examination region is normally coupled with the selection of a measurement program. In the MR apparatuses today, an abundance of measurement programs are supplied by the manufacturer that are normally hierarchically classified. An important sorting criterion is the associability with an anatomical region since some measurement parameters are optimized for the corresponding anatomical region (i.e. a specific examination region). For example, measurement programs for knee examinations are centralized in a measurement program folder with the designation “knee”. The suitable measurement protocols can then be sorted even further dependent on the diagnostic question (such as, for example, meniscus lesion, cartilage damage, . . . ). The information about the associated examination region is normally linked with the selection of a measurement program, and this information enters into the linguistic designation of the produced exposures. These exposures are typically hierarchically organized in a databank, whereby the sorting criterion for the uppermost level is normally the patient name. The information about the examination region, which results from the name of the measurement program, is used as a sorting criterion in a lower level. For some questions, however, no unambiguous designation of the examination region is possible with this procedure. This is the case, for example, given the use of the same measurement program both for the left knee and for the right knee. For unambiguous designation of the examination region, the operator can still input a comment in the control software (for example left knee) which is then graphically shown on the measured exposure.

This typical procedure for determination of examination regions also contains a number of error sources. The manufacturers of MR apparatuses generally give the operator freedom over the sorting and linguistic designation of measurement programs. No logical linguistic designations of measurement programs must be used. Illogical linguistic designations of examination regions can thus also occur. The use of comments is likewise a possible error source. For example, the operator only has to confuse the left knee with the right knee and incorrect image comments are already shown in all measured exposures. The combination with an incorrect specification of the patient position would aggravate this situation even further. The correct patient-related reconstruction of the position and orientation of a measured exposure is then possibly no longer possible.

An exact knowledge of the patient position is therefore likewise of decisive importance for monitoring of an examination region established by the operator. If the patient position is known in detail, this information can be checked from the relative positions of the exposures with regard to the patient.

An objective determination of the patient position is possible in various ways and is of importance not only in MR diagnostics. Patients should be reproducibly positioned and their position monitored in therapeutic radiation therapy. Moreover, optical methods are possible in addition to the positioning by means of mechanical devices such as a displacement table and stereotactic fixing. Examples for this are described in U.S. Pat. Nos. 5,080,100, and 6,279,579 and 5,823,192. They are based on optical measurement systems for three-dimensional surface acquisition or use tracking systems for three-dimensional coordinate detection such as, for example, in the method proposed in U.S. Pat. No. 6,138,302. A transition to the situation in MR examinations is, however, problematic in many aspects. For description of the patient position, the body surface of the patient would have to be measured, which requires an almost completely disrobed patient. This cannot be required in the daily routine. An optical measurement of the patient in the MR apparatus is additionally made significantly more difficult due to the typical tube-shaped construction.

Another possibility of the documentation of the patient position is the reconstruction of the body surface of the patient from a volume data set. The reconstruction of surfaces (among which the body surface also counts) from volume data sets is described in U.S. Pat. Nos. 4,821,213 and 4,719,585. The measurement of volume data sets of the entire patient for detection of the patient position is much to complicated due to the high number of exposures to be produced.

### SUMMARY OF THE INVENTION

An object of the present invention is to provide a method that enables a standardized and objective description of the patient-related position and orientation of exposures in a magnetic resonance tomographic examination. A further object is to provide, a corresponding control device for operation of a magnetic resonance tomography apparatus.

According to the invention, in a method for determination of patient-related information regarding the position and orientation of slice image exposures in magnetic resonance tomographic examinations, initial MR overview exposures (magnetic resonance overview exposures) of the body of the patient are produced. Using these initial MR overview exposures, a predetermined, parameterized anatomical body model (i.e. an anatomical body model) with specific variable model parameters is then individualized (customized). The determination of the patient-related information about the position and orientation of the subsequent (diagnostic) slice image exposures then ensues on the basis of the relative position of the slice image exposures with regard to the individualized body model. In the individualization, by variation of the model parameters the body model is adapted to specific structures determined from the initial MR overview exposures, which specific structures advantageously represent the body surface of the patient. The individualization process thereby corresponds to a mathematical optimization problem. Those values of the variable model parameters are determined that minimize a deviation measure of the model relative to the structures from the overview exposures. This means that an optimally good adaptation of the body model to the entire body of the patient in the actually-present current position essentially ensues.

By the individualization process, information can be transferred from the body model to subsequent diagnostic exposures. A linking of information with the body model located in the normal position (also called “norm model” in the following) is in particular possible, this information also describing the orientation of each part of the norm model in addition to the body region. After the individualization process, both information regarding the position of the exposure in text form and information regarding the orientation of the exposure in the form of orientation markers can be determined from the relative position of an exposure with regard to the parts of the individualized body model located in the immediate neighborhood. This means that, for example, all image pixels or volume elements that exhibit less than a predetermined maximal spatial interval from a region that is defined by a specific body part of the individualized model can be counted in a later MR exposure as components of the appertaining body part of the patient.

An advantage achieved with the invention is that patient-related information regarding the position and orientation of slice image exposures in magnetic resonance tomographic examinations can be determined objectively and in a standardized manner. This includes the automatic generation of orientation markers and text specifications regarding the examination region. An independence is thereby achieved in the determination of the described information by the operator of the MR apparatus, which directly leads to a quality increase of a subsequent medical diagnosis.

In addition to a control interface for activation of the magnetic resonance tomography apparatus for measurement of a number of slice image exposures corresponding to scan parameters predetermined by the control device, and an image data interface for reception of image data acquired by means of the magnetic resonance tomography apparatus, a control device for implementation of the inventive method in the operation of a magnetic resonance tomography apparatus must comprise an overview image determination unit in order to activate the magnetic resonance tomography apparatus for measurement of a number of initial MR overview exposures of the body of the patient. Moreover, the control device has: a storage device with an anatomically parameterized body model, the geometry of which is variable by a modification of specific parameters. The control device also has an individualization unit in order to individualize the body model using the measured initial MR overview exposures, and a localization unit that determines the relative position of the appertaining slice image exposures with regard to the individualized body model for determination of patient-related information about the position and orientation of subsequently-created slice image exposures.

The individualization unit and the localization unit preferably are realized in the form of software on a programmable processor of the control device of a magnetic resonance tomography apparatus. The program memory does not necessary have to be an integrated part of the control device; rather, it is sufficient for the control device to be able to access an external storage device.

In an embodiment of the invention, the initial MR overview exposures are produced in a standardized arrangement. Fast MR sequences thus can be used that are characterized by an acquisition time per overview acquisition in the range of a second. The advantage achieved with this embodiment of the invention is that a uniform examination protocol can be used for acquisition of the overview exposures for each patient. A manual adaptation to the individual patient geometry is not necessary. Moreover, the individualization algorithm is started with a uniform database, which increases the stability of the results.

Cross-section exposures (i.e. exposures oriented transverse to the longitudinal body axis of the patient) are particularly preferably produced as initial MR overview exposures. Cross-section exposures lend themselves to the individualization of a whole-body model since a complete imaging of the body surface is possible in each cross-section exposure. This is generally not the case in exposures along the longitudinal body axis. A complete reconstruction of the body surface of the patient is thus possible in each cross-section exposure given a production of cross-section exposures as initial MR overview exposures. This information increases the stability of the individualization algorithm.

In order to achieve a sufficient model individualization, at least three cross-sections with an interval of approximately 50 cm (given an adult) should be produced as initial MR overview exposures. However, the separation of two adjacent cross-section exposures advantageously lies below 50 cm, particularly preferably even below 15 cm. The more overview exposures that are produced, the more easily the individualization problem can be solved due to the improved database. In practice a compromise must therefore be found between stability and time expenditure. It has been found that a slice separation of approximately 10 cm in the acquisition of the entire patient position represents a good compromise between precision and speed. Moreover, equidistant slice separations do not necessarily have to be selected over the entire body. For example, an acquisition of the hand geometry with the finger positions requires a higher spatial density of overview exposures than the acquisition of the trunk geometry. For example, if a hand is to be examined in detail, a local slice interval of two centimeters can be reasonable. For example, a slice interval of five centimeters is normally sufficient in a foot examination.

It is also possible to initially produce first initial overview images in a more coarse grid. Insofar as the quality of the individualization (which is characterized by the deviation degree to be determined anyway in the individualization) is not sufficient, further overview exposures can be produced at a suitable location. In an embodiment of the invention, the positions and orientations of, if applicable, additional MR overview exposures to be produced are automatically determined via the individualization algorithm given insufficient quality of the individualization. A quantification of the quality is achieved by a calculation of a deviation degree of the body model relative to structures from the overview exposures. This means that the positions and orientations of the additional overview exposures can be determined from the individual algorithm through the analysis of the model deviation relative to structures in the individual overview exposures and the respectively-mapped body region. The advantage achieved with this embodiment of the invention is that, given insufficient quality of the individualization, additional overview exposures are produced with an automatically-running method until a sufficient quality of the individualization is achieved.

In a further embodiment of the invention, the model parameters that are adjustable in an individualization induce at least one translation parameter, one rotation parameter and one scaling parameter of the entire body model as well as further parameters that describe the spatial position and shape of predetermined important body parts such as, for example, the extremities. The number of the parameters for description of the human anatomy primarily depends on the required precision of the modeling. In order to obtain information about the position and orientation of exposures in MR examinations, a precise modeling of anatomical structures internal to the body is of subordinate importance. Rather, a modeling of the movement possibilities of significant body parts and their surface is relevant. A sufficient but not too-detailed modeling of the human anatomy is achieved via the parameterization of the position and shape of the significant body parts.

A relatively simple (but in many situations, sufficient) model can be described, for example, by the following parameters:
body size, arm length, leg length, angle specifications for description of shoulder, elbow, hand, hip, knee and ankle joint, circumference of chest and abdomen.

The following parameters, for example, can be added for a more complex model:
shoulder height, length of upper, lower arm and hand or, respectively, upper, lower leg and foot, angle specifications for description of the head position and of the spinal column divided up into neck, chest and lumbar region as well as the fingers and toes, circumference of head, neck, shoulder, upper arm, lower arm, hand, hips, upper leg, lower leg and foot.

In a further embodiment of the invention, a linguistic designation of the patient position is determined from the parameter values of the individualized model (for example head or feet forward; back, stomach, left side, right side position). The three fundamental model parameters for description of the rotations around the three primary axes are hereby of particularly importance. From these parameters and the further model parameters, conclusions can be made about the linguistic designation of the patient position in the MR apparatus. The difference between the position with the head forward and the position with the feet forward exists in a rotation angle different by 180° around the sagittal axis. The back, stomach, left-side and right-side position is primarily differentiated via the rotation angle around the longitudinal axis. The advantage achieved with this embodiment of the invention is that a linguistic designation of the patient position can be determined objectively and in a standardized manner through the parameter values of the individualized model.

A description of the patient position that is entered as an input by the operator preferably is monitored with the aid of the parameter values of the individualized model. Insofar as the linguistic designations of the patient position that are possible with the individualization algorithm coincide with the selection possibilities of the patient position description by the operator, the description of the patient position that is entered by the operator can be automatically monitored. A quality increase of the examination is achieved by such a monitoring of the patient position description using the individualization algorithm.

In a preferred embodiment of the invention, the patient-related information about the position and orientation of slice image exposures is encoded in linguistic and/or graphical form and represented with the individualized model. Patient-related information (for example by means of text specifications and orientation markers) about the position and orientation of slice images are normally represented in the individual exposures. For better clarity the individual exposures can also be represented three-dimensionally in their position and orientation with the individualized body model. The advantage achieved with this embodiment of the invention is that, in addition to the patient position, the position of the exposures with regard to the patient can also be graphically represented in a simple and detailed manner with this method.

The body weight of the patient can be calculated using the individualized body model. A body weight input by the operator or already present in a patient data file can be monitored. The body weight of the patient is important for calculation of the specific absorption rate (SAR). The volume (and from this the body weight of the patient) can be estimated from the individualized body model. An additional quality increase of the examination is consequently achieved the monitoring of the body weight using the individualization algorithm. In particular the SAR limit values are reliably maintained.

In a further embodiment of the invention, a positioning of the patient in the MR apparatus for examination of a desired region is implemented with the aid of the individualized model. The method for determination of information regarding the position of exposures is reversed for this purpose. An examination region is then provided and a suitable initial position for subsequent exposures is sought. For this purpose, those parts of the individualized model with which the desired region is linked are determined for the desired examination region. A table displacement that brings the examination region into the magnetic field center is calculated from the spatial position of these parts. This procedure enables an automatic positioning of the patient dependent on the desired examination region. A time-consuming manual positioning of the region to be examined, which is normally implemented with laser light beam localizers, can then be omitted.

A model individualized in a first MR examination is preferably stored and a positioning of the patient in a further MR examination is implemented with the aid of this individualized body model. In this manner it can be ensured that the patient optimally assumes exactly the same position in a later MR progressive examination an in the first MR examination. The adoption of the same patient position is of decisive importance given a progressive examination. In particular the joint positions and the shape of the soft parts are dependent on the positioning. Not only is a qualitative comparison is by the comparison of a model individualized in the subsequent progressive examination with the stored individualized model, but also a degree of deviation of both models can be calculated, and this can be used for adoption of the same patient position. The sum of the squares of deviation of corresponding model triangles is suitable as a measure of deviation of both different individualized models. This definition is unambiguous since the two form-variable models differ only by the spatial positions of the model triangles, and the triangle count as well as the neighborhood relationships of the triangles remain unaltered. A quality increase of the examination is achieved by the manageable adoption of an optimally identical patient position in an MR progressive examination.

A signal is emitted in the case of an incorrect process and/or at the end of one or all method steps. When a notification of the operator ensues via a signal given automatically executing method steps, the operator can make use of this or her time for other tasks. This increases the workflow in the examination.

### DESCRIPTION OF THE DRAWINGS

FIG. 1 is a schematic representation of the primary coordinate system and the primary planes of the body of a patient.

FIG. 2 is a flowchart of an embodiment of the inventive method.

FIG. 3A is a schematic representation of a patient in the normal position on a patient bed, lying on his back.

FIG. 3B is a schematic representation of a patient in a position deviating from the normal position, lying on his back.

FIG. 4 is a schematic representation of a magnetic resonance tomograph with an exemplary embodiment of an inventive control device.

### DESCRIPTION OF THE PREFERRED EMBODIMENTS

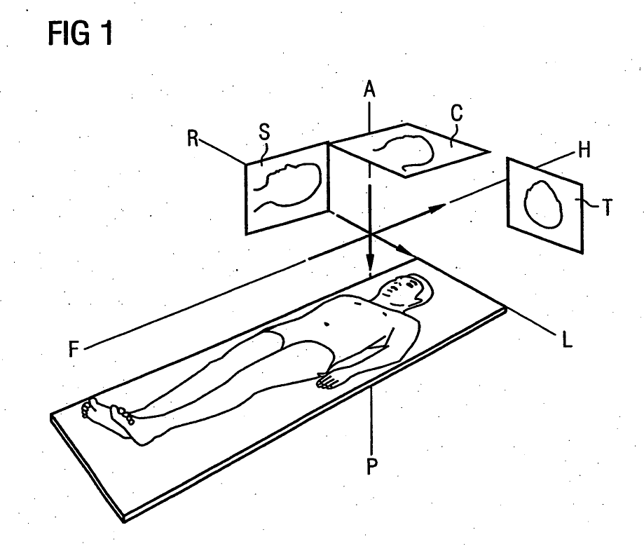

As explained above, FIG. 1 illustrates the primary coordinate system of the body of a patient in the normal position.

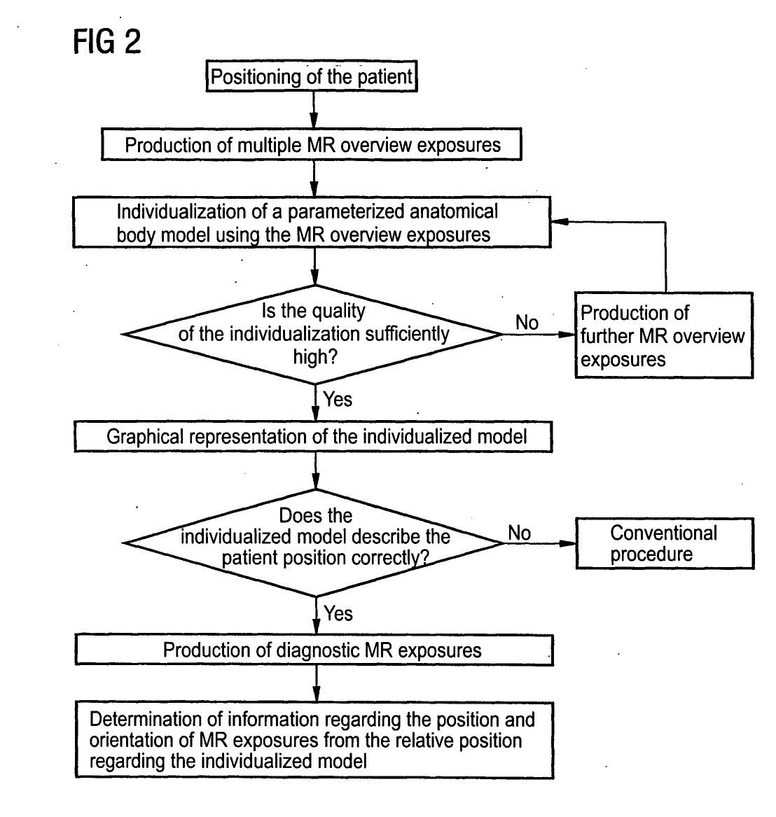

An exemplary workflow of the inventive method as shown in FIG. 2 includes the following steps after the positioning of the patient:

a.) Production of a number of initial MR overview exposures of the patient.

b.) Individualization of a parameterized anatomical body model on the basis of the produced initial MR overview exposures.

c.) Given insufficient quality of the individualization, additional MR overview exposures are produced and the individualization method is re-implemented.

d.) Graphical representation of the individualized model for method monitoring.

e.) The method is interrupted by the operator given insufficient description of the patient position by the individualized model.

f.) Production of diagnostic MR exposures.

g.) Determination of information regarding the position and orientation of MR exposures from the relative position with regard to the individualized model.

For model individualization, cross-section exposures in standardized arrangement thereby lend themselves as initial overview exposures since a cohesive reconstruction of the body surface is then possible in the individual cross-section exposures. A single threshold method can be used for this, whereby the jump in the signal intensity from air to skin defining the body surface. Due to the large geometric measurement regions of the MR apparatuses, it can normally be assumed that complete cross-sections of the patient are acquired in the measurement. This leads to a contiguous reconstruction of the body surface in the individual cross-section exposures that is mathematically expressed in the description of the body surface via closed continuous lines. These form the target structures in the individualization.

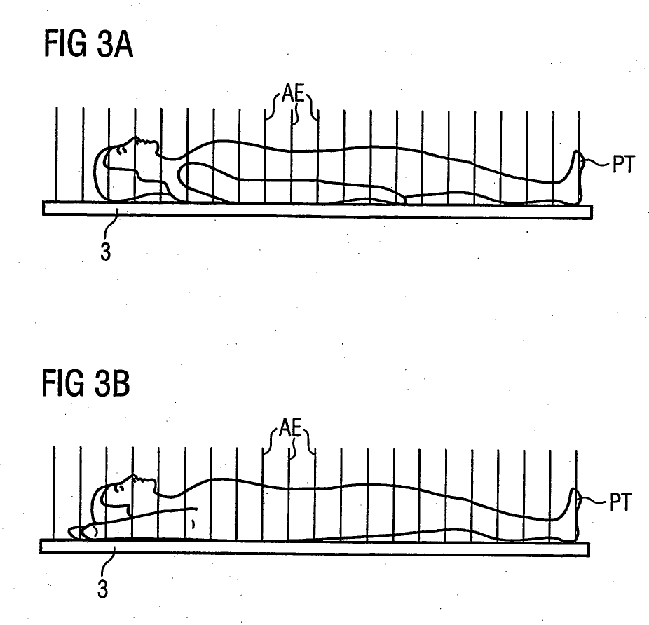

FIGS. 3A and 3B respectively show a patient PT lying on his back in the normal position (FIG. 3A) on a patient positioning table 3 and deviating from the normal position in a position (FIG. 3B), likewise lying on his back, however with the right hand over the head. Moreover, the exposure planes AE are shown in which the initial cross-section overview exposures are produced. The separation of the exposure planes here is approximately 10 cm. FIGS. 3A and 3B illustrate that, given corresponding separation of the cross-section exposures relative to one another, a positioning of an extremity of the patient PT that does not correspond to the normal position is acquired well, and this posture correspondingly, ultimately, correctly reproduces the individualized body model.

In addition to the target structures, a suitable body model is still required for the individualization process, whereby the variable parameters of said body model are not unambiguously determined. Only the underlying parameters for the elementary transformations such as the translation, the rotation and the scaling of the body model are clear. In contrast to this, the type and number of the parameters describing the model geometry primary depend on the usage purpose.

In order to acquire information about the position and orientation of exposures in MR examinations, a modeling of the movement possibilities of significant body parts and their surface is primarily relevant. A design of the body model from a number of contiguous volume elements thereby is suitable, their dimensions lying in the centimeter range, and the concrete model geometry is determined by a set of parameter values. Suitable methods are described in the article “Simulating facial surgery using finite element models” by Koch et. al. (Proceedings of the SIGGRAPH 1996 conference, p. 421-428) for a finite element model or in the article “A 3D anatomical atlas based on a volume model”by Höhne et. al. (IEEE Computer Graphics Applications, 1992, volume 12, nr. 4, p. 72-78) for a voxel model.

Very simple body models can be described with only one pair of parameters such as, for example, body size, arm length, leg length, chest circumference, abdomen circumference as well as parameters for description of the arm and leg position.

Complex body models with a large parameter count are juxtaposed with this. Not only the body surface but also sometimes the skeleton system can be modeled. The geometry of the human back is thus, for example, described by the following parameters: Distantia cristarum, Distantia spinarum, Diameter spinarum posterior, Diameter transversa of the pelvic width, Diameter transversa of the pelvic strait, Diameter transversa of the pelvic outlet, Diameter sagittalis of the pelvic width, Diameter sagittalis pelvic strait, Diameter sagittalis pelvic outlet, Conjugata anatomica, Conjugata diagonalis, Conjugata vera.

In order to obtain information about the position and orientation of exposures in MR examinations, however, such a precise modeling normally is not necessary. Depending on the desired precision, a modeling of the significant body parts is sufficient. The position of the body parts is thereby primarily described by the degree of freedom of the anatomical joints. For example, in general three parameters (namely dilation and deflection, abduction and adduction, inner rotation and outer rotation are sufficient for description of the hip joint (which is a ball joint). The shape of a femur can be parameterized, for example, by two respective diameters arranged at the start, in the middle and at the end of the femur and situated perpendicular to one another, which diameters represent the primary axes of the (in good approximation) oval femur. One can also similarly proceed with the other body parts such as head, neck, chest, abdomen, back, shoulder girdle, upper arm, lower arm, hand, lower leg, foot. At least the movement possibilities of the large joints of the human body should be parameterized. The relevant joints are: upper and lower ankle, knee joint, hip joint, shoulder joint, elbow joint, wrist.

The spinal column is an exceptional case. Because the spinal column is composed of multiple joints between the individual vertebrae, the movement possibilities can be parameterized in detail only with effort. A sub-division of the spinal column into the neck, chest and lumbar spinal column regions with simplified movement possibilities is reasonable for the description of the patient position.

In examinations of the finger that occur quite seldom, another additional parameterization of the movement possibilities of the finger joint is reasonable.

The value range of the individual parameters is advantageously limited. The limitation ensues such that only parameter value combinations are allowed that describe an anatomically-possible position of the patient. The parameter values belonging to the normal position of the model (norm model) are also designated as normal parameter values.

The individualization algorithm can then be started with the described target structures in the individual overview exposures and the body model. The goal is the determination of a set of parameter values that minimize the deviation of the body model relative to the target structures on the overview exposures. The individualized body model with the individualized parameter value set is obtained as a result.

The deviation of the body model relative to the target structures is described by a deviation function. The arguments of the deviation function are the model parameter values. Mathematically, a nonlinear optimization problem exists, namely minimization of the deviation function. Each allowed parameter value combination leads to a body model with specific geometry, which body model is made up of volume elements. One possible deviation function can then be defined by the deviation values of the surface elements of the body model. The calculation of the deviation as a weighted sum of the squares of the deviation values from the individual surface elements thereby volunteers itself. The weighting factor of a surface element is the ratio of the area of the surface element to the average value of the areal contents of all surface elements. This way of deviation calculation via the use of the model surface is reasonable since the target structures are parts of the body surface of the patient.

The minimum geometric separation from the target structures is defined as a deviation value of a surface element, insofar as the surface element geometrically intersects an overview exposure. Otherwise, the deviation value is not defined. This is reasonable since no information about the separation relative to the body surface of the patient exists anyway for a surface element situated in the middle between two overview exposures. A different number of surface elements thus enter into the deviation calculation depending on the position of the body model. In order to nevertheless be able to calculate comparable deviations from these values for different parameters, a deviation normalization of the area of all surface elements incorporated into the calculation is reasonable. The optimal parameter set for minimization of the deviation can then be determined via conventional search path or raster methods. Suitable methods are, for example, described in “Numerische Mathematik”, R. Schaback, Springer Verlag, 1992.

The quality of the individualization is quantified by the value of the calculated deviation. If this lies below a predetermined limit value, the individualization has been successfully implemented. When it does not, further overview exposures are then produced at points with the largest deviation values from the surface elements to increase the adaptation quality. The body surface of the patient is thus exactly dimensioned at the critical regions determined by the individualization algorithm. This procedure is iteratively run through until a sufficiently exact description of the patient position is achieved by the body model or it is interrupted after a certain iteration number given insufficient convergence behavior.

The three-dimensional graphical representation of the individualized body model enables the operator a control of the individualization via the comparison with the actual patient positioning. Upon the occurrence of relevant deviations, the operator can abort the method and continue working conventionally.

Given successful individualization, patient-related information regarding the position and orientation of the exposures can be determined from the relative position of the subsequent diagnostic exposures with regard to the individualized body model. For this purpose, information is linked with each volume element of the norm model before the individualization. This information sometimes describes the body region and orientation of each volume element. For example, the information “right knee” in text form is linked with all volume elements of the norm model that form the right knee. Information in text form regarding the position of the exposure can then be acquired after the individualization process from the relative position of an exposure with regard to the associated volume elements of the individualized body model. If, for example, the exposure intersects only volume elements with the linked information “right knee”, then “right knee” can also be specified as an examination region of the exposure (i.e. as position information regarding the exposure). The information regarding the body region would thus transfer from the individualized body model to the exposure and there designate the examination region.

The same is with orientation markers. The orientation of a volume element is described by a local coordinate system, whereby in a normal position all local coordinate systems coincide with the primary coordinate system. After the individualization, these normally no longer coincide. If, for example, the patient is positioned with the arms over the head, after the individualization the local coordinate systems in the volume elements of the hand describe the local orientation axes and differ from the local orientation axes at the torso. If an exposure then images the hand of the patient, information regarding the orientation of the exposure can then be acquired from the relative position of the exposure with regard to the corresponding volume elements of the hand. The orientation markers of the exposure then locally refer to the hand and no longer refer to a uniform coordinate system for the entire patient.

A linguistic designation of the patient position can also be acquired as an ancillary product from the parameter values of the individualized body model (for example head or feet forward, back, abdomen, left side, right side position), whereby the three parameters for description of the rotation around the three primary axes have a prominent importance. For example, the back, abdomen, left-side and right-side positions primarily differ by the rotation angle around the longitudinal axis. In contrast to this, the positioning of the arms is primarily described by the parameters for description of the shoulder as well as elbow joints. A linguistic designation of the patient position can be associated with each set of parameter values. In practice, a linguistic designation of the patient position in tabular form can be defined for an endless number of parameter value sets. For an arbitrary parameter value set, the parameter value set corresponding best is then determined from the table with associated linguistic designation of the patient position and displayed to the operator in text form. The patient position description in text form also enables a simple monitoring of the description of the patient position selected by the operator, insofar as in both cases the same pool of linguistic designations was used.

Patient-related information about the position and orientation of exposures are normally graphically represented in the exposures, for example via text specifications and orientation markers. In addition to this two-dimensional representation form, a three-dimensional visualization of the individual exposures with the individualized body model volunteers itself. Various 3D representation techniques can be used for this. For example, hardware-accelerated methods on a triangle basis (SSD method, Surface Shaded Display) as it is described in the “OpenGL Programming Guide”, Woo et al., Addison Wesley publishing, 3rd edition, 1999 lend themselves to real-time visualizations. This visualization technique enables an interactive observation of the scenery in real time and nevertheless possess a sufficient representation quality. A simple as well as intuitive representation of the significant information is achieved via simultaneous visualization of the triangulated model surface with the produced exposures in their three-dimensional position and orientation. The text specifications and orientation markers can be mapped as well.

Further information can also be extracted from the individualized body model in addition to the information regarding the position and orientation of exposures. The estimation of the body weight of the patient is quite simple. A density is associated with each volume element of the body model before the individualization. After the individualization, the weight of the volume element is calculated from this information and the volume of the volume element. The sum of the weights of the individual volume elements then yields the estimated value of the patient weight.

Other information enables an automatic positioning of the patient for examination of a desired body region in the MR apparatus. This method uses the information about the associated body region, which information is linked with each volume element. For this purpose, those volume elements of the individualized body model with which the linguistic designation of the region is linked are determined for the desired examination region. The geometric center of these volume elements defines the center of the examination region and is brought into the magnetic. field center via a then-defined table displacement. For example, with one patient it is desired to examine the right knee. After the individualization, the positions of volume elements with the linked information “right knee” are known. The center point of these volume elements then defines the position with regard to the examination of the knee. The table displacement results as a difference between magnetic field center and the calculated center point.

A further application possibility of individualized body models exists in the adoption of the same patient position in progressive examinations. For this, the model parameter values of the reference examination are stored and re-imported in a progressive examination. These parameter values then define the reference model. For adoption of the same patient position, the individualized body model of the progressive examination (progressive model) is compared with the reference model. The goal is a deviation minimization of the two body models. For this purpose, a deviation value is associated with each surface element of the progressive model, this deviation value being defined as a geometric distance from the corresponding surface element of the reference model. The deviation is defined as a sum of the squares of the deviation values. The patient position is varied in the progressive examination until the respectively newly-determined progressive model shows a sufficient correlation with the reference model. A simultaneous three-dimensional representation of both models with a color coding of the deviation values is helpful for this.

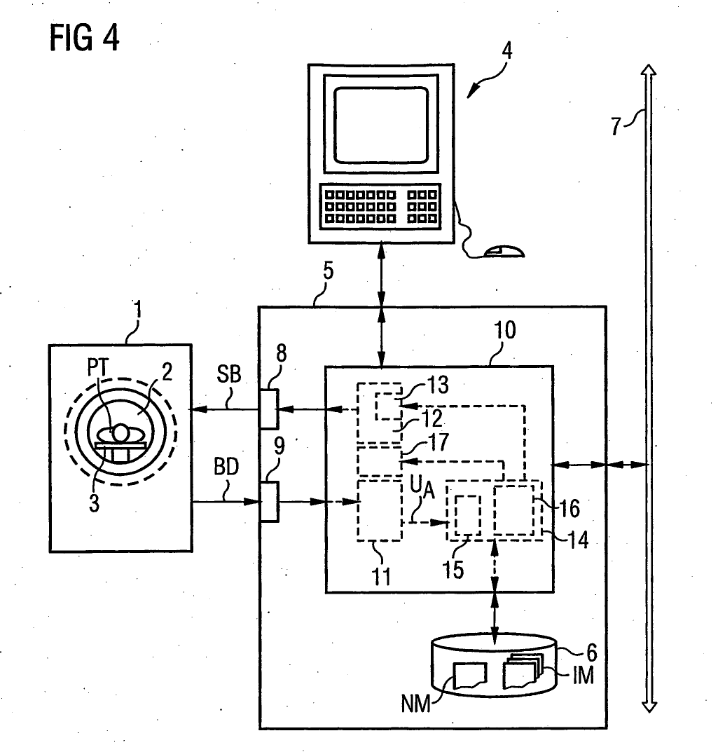

An exemplary embodiment of an inventive magnetic resonance tomography apparatus 1 with an associated inventive control device 5 is shown schematized in FIG. 4.

In the shown exemplary embodiment, the control device 5 is housed in a separate apparatus. This is a computer with a programmable processor 10 on which is stored the control software for activation of the magnetic resonance tomography apparatus 1. The control device 5 transmits control commands SB to the magnetic resonance tomography apparatus 1 via a control interface 8 so that the desired measurement is implemented by this magnetic resonance tomography apparatus 1. The raw image data BD acquired by means of the magnetic resonance tomography apparatus 1 are transferred via the image data interface 9 and then further-processed within the control device 5 in a typical manner.

In order to be able to operate the control device 5, it is connected to a console 4 which, as, a user interface, comprises a screen, a keyboard and a pointer device (for example a mouse). It is alternatively also possible that, instead of ensuing via the console 4 directly connected to the control device 5, the operation ensues, for example, via a workstation (not shown) which is connected to a bus 7 with which the control device 5 is connected. The console 4 can also be an integrative component of the control device 5. The control device 5 can likewise also be an integrative component of the magnetic resonance tomography apparatus 1, such that all components are comprised in one apparatus.

The magnetic resonance tomography apparatus 1 is here a conventional magnetic resonance tomography apparatus with typical radio-frequency, gradient and basic magnetic field coils (not shown). The patient PT is positioned in the magnetic resonance tomography apparatus 1 on a patient positioning table 3 within a measurement chamber 2 around which the coils are arranged. Local coils can additionally be used that are positioned directly on the patient PT. The features and the functionality of a magnetic resonance tomography apparatus 1 are known to those skilled in the art and need not be explained further herein.

The core of the control device 5 is a processor 10 on which various components are implemented in the form of software so that the control device 5 functions in the inventive manner. These components are schematically represented in FIG. 4 as blocks within the processor 10. In addition to the shown components, the control device 5 naturally has all further typical software and hardware components in order to be able to control a magnetic resonance tomography apparatus in a typical manner and acquire and (pre-)process image data. For better clarity these typical components are not shown in FIG. 4 and are not explained in detail in the following insofar as they were not specially modified for the inventive function.

Such a component is an image determination unit 12. This image determination unit 12 converts various measurement protocols or, respectively, scan parameters thereby predetermined (with which measurement protocols or, respectively, scan parameters it is signaled to the magnetic resonance tomography apparatus 1 in which position or, respectively, orientation image data should be determined) into control commands SB. These are then transferred to the magnetic resonance tomography apparatus 1 via the control interface 8 so that there the matching measurement sequences are conducted in the correct order in order to generate the desired slice images. For realization of the invention, the image determination unit 12 has an overview image determination unit 13 (here as a subroutine) which ensures that the magnetic resonance tomography apparatus is controlled such that a number of initial MR overview exposures (such as, for example, shown in FIGS. 3a and 3b) are measured.

The MR overview exposures generated in these overview scans are then (like all remaining image data BD) transferred from the control device 5 via the image data interface 9 and are further processed therein. For example, in a reconstruction unit 11 the desired images are initially reconstructed from the determined raw image data BD. The MR overview exposures UA so determined are then transferred to an individualization unit 14.

This individualization unit 14 contains a structure detection unit 15 as a subroutine which determines the necessary structures (here the boundary surfaces between the body of the patient PT and the environment, i.e. the surface structure of the body of the patient PT) from the overview exposures UA. The individualization unit 14 additionally contains an adaptation unit 16 which adapts a norm model NM to the target structure via adjustment of specific, variable parameters of the norm model NM (i.e. a body model in normal position) as this was already described in detail above. Such a norm model NM is stored in a storage 6 of the control device 5.

The finished, individualized body model IM can then again be stored in the storage 6. All data that describe the complete, individualized body model IM do not necessarily thereby have to be stored. In principle it is sufficient when a set of parameter values is stored with which the individualized body model IM can be generated from the norm model NM. The data set of the individualized body model IM is moreover transferred to a localization unit 17. The relative position of the subsequent (diagnostic) slice image exposures with regard to the individualized body model IM can then respectively be determined with the aid of this localization unit 17, and the patient-related information about a position and orientation of the subsequently generated slice image exposures can thus be determined in the inventive manner.

Moreover, the individualization unit 14 is able to output a signal to the overview images determination unit 12 in order to ensure that a larger number of overview images situated more closely to one another are additionally produced in specific spatial regions in order to improve the quality of the individualization.

The processes and system architectures shown in FIGS. 1-4 are only exemplary embodiments, and can be modified in detail without further measures by those skilled in the art. In particular it is possible that most components of the control device 5 are realized not on a processor but rather on various processors networked among one another. Naturally, it is likewise also possible that the various components are realized on different computers networked with one another. For example, particularly calculation-intensive processes such as the individualization of the model can thus be sourced out to via the bus 7, which suitable computers then deliver back only the end result.

Moreover, existing control devices magnetic resonance tomography apparatuses can be retrofitted with the inventive components. In many cases, an update of the control software with suitable control software modules is sufficient.

Although modifications and changes may be suggested by those skilled in the art, it is the invention of the inventor to embody within the patent warranted heron all changes and modifications as reasonably and properly come within the scope of his contribution to the art.

I claim as my invention:

1-15. (canceled)

16. A method for determining information regarding position and orientation of magnetic resonance tomographic slice image exposures of a patient, reference to the patient, comprising the steps of:
generating a plurality of initial MR overview exposures of the body of a patient;
electronically individualizing electronic data representing a predetermined, parameterized anatomical body model using said initial magnetic resonance overview exposures; and
automatically electronically determining patient-referenced information indicating a position and orientation of subsequent slice image exposures of the patient, obtained after said initial MR overview images, dependent on a relative position of said subsequent slice image exposures with respect to the individualized body model.

17. A method as claimed in claim 16 comprising producing said initial magnetic resonance overview exposures in a standardized arrangement.

18. A method as claimed in claim 16 comprising generating said initial magnetic resonance overview exposures as cross-section exposures of the patient.

19. A method as claimed in claim 18 comprising generating said cross-section exposures, comprising said initial magnetic resonance overview exposures, as a plurality of cross-section exposures with respective intervals therebetween of no greater than approximately 50 cm.

20. A method as claimed in claim 18 comprising generating said cross-section exposures, comprising said initial magnetic resonance overview exposures, as a plurality of cross-section exposures with respective intervals therebetween of no greater than approximately 15 cm.

21. A method as claimed in claim 16 comprising automatically electronically determining a quality of individualization of said body model by individualizing said anatomical body model in successive iterations and, after each iteration, comparing the individualized body model to a structure therein that is also detectable in said initial magnetic resonance overview exposures.

22. A method as claimed in claim 16 comprising individualizing said body model by adjusting model parameters comprising at least one translation parameter, at least one rotation parameter and at least one scaling parameter of an entirety of the body model, in addition to parameters describing a spatial position and shape of predetermined body parts of said body model.

23. A method as claimed in claim 16 comprising determining a linguistic destination of the position of the patient using parameter values of said individualized body model.

24. A method as claimed in claim 16 comprising automatically positioning said patient dependent on a patient description entered by an operator, and automatically electronically monitoring a description of said patient position using parameter values of said individualized body model.

25. A method as claimed in claim 16 comprising providing a visualizeable output of said position and orientation of said subsequent slice image exposures dependent on said individualized body model.

26. A method as claimed in claim 25 comprising providing said visualized output in a form selected from the group consisting of a linguistic form and a graphical form.

27. A method as claimed in claim 16 comprising using said individualized body model to automatically electronically calculate a body weight of the patient.

28. A method as claimed in claim 16 comprising automatically electronically positioning the patient relative to a magnetic resonance scanner, for obtaining said subsequent magnetic resonance exposures, using said individualized body model.

29. A method as claimed in claim 16 comprising electronically storing said individualized body model, and generating said subsequent magnetic resonance images of the patient at a time substantially separated from a time at which said initial magnetic resonance images of the patient were generated, with electronic access to the stored individualized body model.

30. A computer readable medium encoded with information in computer readable form that programs a computer to operate a magnetic resonance imaging apparatus to:
generating a plurality of initial MR overview exposures of the body of a patient;
electronically individualizing electronic data representing a predetermined, parameterized anatomical body model using said initial magnetic resonance overview exposures; and
automatically electronically determining patient-referenced information indicating a position and orientation of subsequent slice image exposures of the patient, obtained after said initial MR overview images, dependent on a relative position of said subsequent slice image exposures with respect to the individualized body model.

31. A control device for operating a magnetic resonance tomography apparatus having a scanner adapted to receive a patient therein, said control device being programmed to:
generating a plurality of initial MR overview exposures of the body of a patient;
electronically individualizing electronic data representing a predetermined, parameterized anatomical body model using said initial magnetic resonance overview exposures; and
automatically electronically determining patient-referenced information indicating a position and orientation of subsequent slice image exposures of the patient, obtained after said initial MR overview images, dependent on a relative position of said subsequent slice image exposures with respect to the individualized body model.

32. A magnetic resonance apparatus comprising:
generating a plurality of initial MR overview exposures of the body of a patient;
electronically individualizing electronic data representing a predetermined, parameterized anatomical body model using said initial magnetic resonance overview exposures; and
automatically electronically determining patient-referenced information indicating a position and orientation of subsequent slice image exposures of the patient, obtained after said initial MR overview images, dependent on a relative position of said subsequent slice image exposures with respect to the individualized body model.
